(19) United States
(12) Patent Application Publication
(10) Pub. No.: US 2017/0087437 A1
(43) Pub. Date: Mar. 30, 2017

(54) EXERCISING PACING DEVICE AND METHODS OF OPERATION

(71) Applicant: BV Systems, LLC, Shawnee, KS (US)

(72) Inventors: Barry Vonada, Shawnee, KS (US); Damian Smithhisler, Andale, KS (US); Dennis Smithhisler, Wichita, KS (US)

(21) Appl. No.: 15/235,742

(22) Filed: Aug. 12, 2016

Related U.S. Application Data
(60) Provisional application No. 62/301,984, filed on Mar. 1, 2016.
(60) Provisional application No. 62/235,297, filed on Sep. 30, 2015.

(51) Int. Cl. A63B 71/06; A63B 69/00; A61B 5/024

(52) U.S. Cl. CPC A63B 71/0686; A61B 5/02438; A63B 69/0028; A63B 2230/062; A63B 2225/50; A63B 2071/0694

(57) ABSTRACT

An exercise pacing device includes multiple lights that are configured to be placed at regular intervals along a running track. The lights are controlled by a controller to turn on and off along the continuous loop of the elongated structure. The lights are turned on and off at a speed sufficient to simulate a moving indicator for pacing a user around the running track.

### RELATED APPLICATIONS

This application relates to and claims the priority of U.S. Provisional Patent Application No. 62/235,297, which was filed on Sep. 30, 2015 and U.S. Provisional Application No. 62/301,984, which was filed on Mar. 1, 2016, each of which is hereby incorporated by reference in its entirety.

### FIELD OF INVENTION

The present invention generally relates to exercise devices, and more particularly, to an exercise pacing device and a method of its operation.

### BACKGROUND

Athletic training often involves running. Owing to its popularity, athletic venues, typically referred to as track and field events, have been created where contestants (e.g., athletes) compete with one another to test their running skills against one another. These track and field events often involve sprint competition as well as long-distance competition. Long-distance running, or endurance running, is a form of continuous running over long distances. Physiologically, it is largely aerobic in nature and requires stamina as well as mental strength. In contrast to sprint competition, contestants of long-distance running often pace themselves in order to conserve sufficient energy needed for completion of these long-distance running events.

### SUMMARY

According to one embodiment, an exercise pacing device includes multiple lights that are configured to be placed at regular intervals along a running track. The lights are controlled by a controller to turn on and off along the continuous loop of the elongated structure. The lights are turned on and off at a speed sufficient to simulate a moving indicator for pacing a user around the running track.

### BRIEF DESCRIPTION OF THE DRAWINGS

The foregoing and other objects, features and advantages of the disclosure will be apparent from the following description of particular embodiments of the disclosure, as illustrated in the accompanying drawings in which like reference characters refer to the same parts throughout the different views. The drawings are not necessarily to scale, emphasis instead being placed upon illustrating the principles of the disclosure.

FIG. 1 illustrates an example exercise pacing device according to one embodiment of the present disclosure.

FIGS. 2A, 2B, and 2C illustrate example modular elongated sections that may be used to construct the elongated structure according to one embodiment of the present disclosure.

FIG. 3 illustrates an example elongated section and associated circuitry that may be used to generate the moving indicator through its respective section according to one embodiment of the present disclosure.

FIGS. 4A and 4B illustrate a perspective view and end view, respectively, of an extruded member according to one embodiment of the present disclosure.

FIGS. 5A, 5B, and 5C illustrate a perspective view, a side view, and a top view, respectively, of a flex coupling according to one embodiment of the present disclosure.

FIG. 6 illustrates one example of the pacing device controller that may be used to control the operation of the exercise pacing device according to one embodiment of the present disclosure.

FIG. 7 illustrates an example process that may be performed by the exercise pacing device according to one embodiment of the present disclosure.

FIG. 8 illustrates a block diagram of an example computer device for use with the example embodiments of the present disclosure.

### DETAILED DESCRIPTION

Athletes who train for sprint and long distance running often use paced training sessions (e.g., interval training), which often requires the athlete to run a prescribed distance at a specific pace or time. In many cases, this has to be achieved by estimating the pace as there has heretofore been no visual aid or guide that keeps the athlete on pace. Conventionally, the athlete has had to rely on guesswork (e.g., feel) to achieve the proper pace for the distance workout. Additionally, a coach or a partner would often have to verbally convey pacing information to the athlete while the athlete ran along a running track so that the athlete could adjust his or her speed to maintain the properly prescribed pace. Thus, there has heretofore remained a long felt need for a system that paces an athlete along a track or other running surface in a consistent and efficient manner.

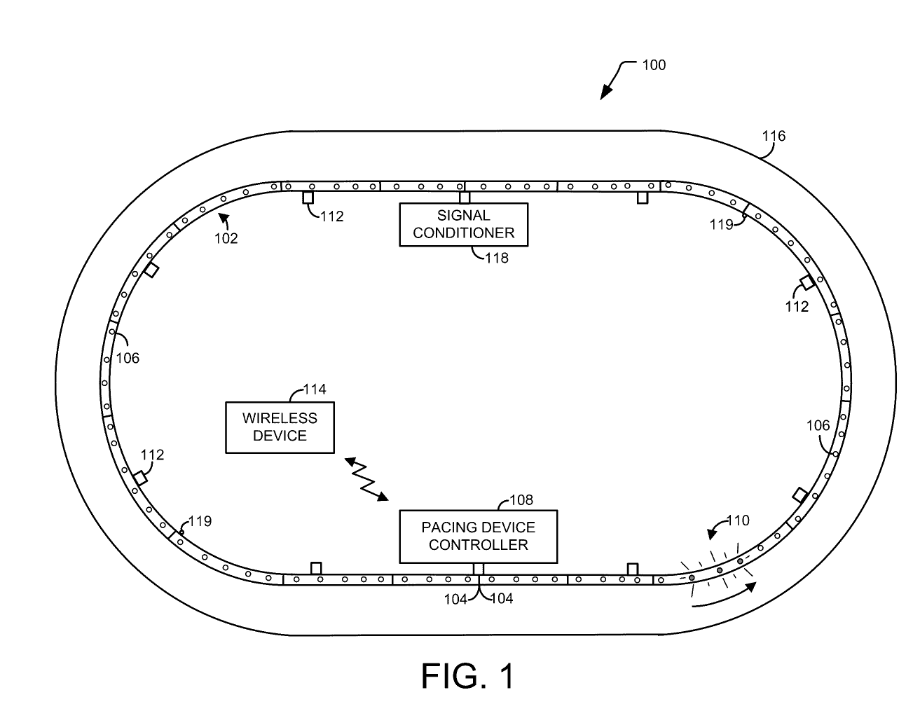

FIG. 1 illustrates an example exercise pacing device 100 according to one embodiment of the present disclosure. The exercise pacing device 100 includes an elongated structure 102 having two ends 104 that are configured to be disposed adjacent one another to form a continuous loop. The elongated structure 102 includes multiple lights 106 configured at regular intervals along the elongated structure 102. The exercise pacing device 100 also includes a pacing device controller 108 to control the lights 106 to turn on and off in a sequential fashion along the extent of the elongated structure 102 and at a speed sufficient to simulate a moving indicator 110 for pacing a user (e.g., athlete) around the continuous loop. The exercise pacing device 100 also includes one or more sensors 112 that can be used to obtain information, such as proximity information associated with the user, and/or telemetry information associated with one or more performance characteristics of the user.

The pacing device controller 108 may be controlled by a wireless device 114, such as a cellular telephone or a tablet, which transmits commands to the pacing device controller 108 and may optionally receive telemetry information back from the pacing device controller 108, such as information associated with the operational health of the pacing device controller 108 and/or elongated structure 102. In one embodiment, the cellular telephone or tablet may be installed with a custom application (e.g., a mobile application) that is configured to communicate with the pacing device controller 108 for controlling its operation using a suitable communication medium (e.g., Bluetooth signaling). The wireless device 114 includes instructions stored in a memory and executed by one or more processors for performing the various features of the wireless device 114 described herein.

Generally, the pacing device controller 108 energizes the lights 106 to turn on and off in sequential fashion along the elongated structure 102 such that the energized lights 106 generates a moving indicator 110 that mimics or simulates the action of a rabbit typically used at a racing track (e.g., a dog racing track). The lights 106 may be any suitable type, such as light emitting diodes (LEDs) that emit similar or different colors of light relative to one another. Nevertheless, it is contemplated that other forms of indicators may be implemented to guide the user around the elongated structure 102. For example, it is contemplated that one or more auditory chirping devices may be disposed around the elongated structure 102 that generate a chirping sound for pacing the user around the elongated structure 102.

In one embodiment, the pacing device controller 108 energizes a specified number of adjacent lights at one time. For example, the pacing device controller 108 may initially energize the first, second, third, and fourth lights along the elongated structure 102, energize the second, third, fourth, and fifth lights along the elongated structure 102 after a specified period of time, and again energize the third, fourth, fifth, and sixth lights along the elongated structure after a second instance of the specified period of time. This process may be repeated along the elongated structure continually for as many laps around the elongated structure 102 that are desired by the user.

The controller 108 may be programmed to control the moving indicator 110 in any suitable manner. For example, the controller 108 may be programmed to create a training session that includes one or more parameters, such as a desired pace (e.g., speed), a length of the training session (e.g., quantity of laps), changes in the speed of the moving indicator 110 during the training session, sensor information to be obtained and correlated with each use of the training session, and the like. The created training session is then stored in memory for repeated use in the future. In some embodiments, the controller 108 may be programmed to control the moving indicator 110 without saving and retrieving parameters from memory. For example, the controller 108 may be programmed with only a speed for the moving indicator 110 such that when a starting signal is received, the controller 108 controls the moving indicator 110 to move around the elongated structure 102 for an indefinite period of time or until the controller 108 is turned off.

Nevertheless, when the controller 108 receives a start signal, it controls the moving indicator 110 to move around the elongated structure 102 such that a user (e.g., athlete) may run in stride with the illuminated moving indicator 110. For example, the pacing device controller 108 may be programmed to advance the moving indicator 110 over a distance of 200 meters within 35 seconds. Without any visual feedback, the user may run too fast or too slow and not meet the required time. However, with the exercise pacing device 100, the user may run in stride with the illuminated moving indicator 110 so that the specified time may be adequately met.

The pacing device controller 108 interprets user input provided from the wireless device 114 and performs one or more algorithms to drive the lights 106 (e.g., light emitting diodes) according to the user input. For example, the wireless device 114 may accept user (e.g., coach, trainer, etc.) input regarding a running distance and a time (e.g., pace). That information is transmitted wirelessly to the pacing device controller 108, which is then fed to the processing system for translation. The translation converts the user input data into clocking signals that will propagate the moving indicator 110 along the elongated structure 102. The user may also use the wireless device 114 start, stop, and/or pause the system in a similar manner in which a stopwatch or other timing device is used. In one embodiment, one or both of the wireless device 114, pacing device controller 108, and signal conditioner 118 may be powered by rechargeable batteries. In use, the pacing device controller 108 will cause a first segment of lights 106 to illuminate briefly before turning off and subsequently illuminating a second segment of lights 106. This sequence will continue at a rate matching the user's desired time or pace. The interpretation and conversion of this data within the pacing device controller 108 is a key function of the processing system.

Any user, such as a coach or trainer, could use the exercise pacing device 100 to provide athletes with a visual feedback, timing device. Additionally, use of the exercise pacing device 100 may enhance confidence that a training regimen with a set speed and distance is being adhered to. Athletes who train at high levels typically rely on specific workouts that will build and enhance specific physiological aspects of their body. Embodiments of the present disclosure provide an exercise pacing device 100 that may alleviate at least some of the guesswork from those workouts. Embodiments are described herein for use with the regimen of running; however, it is contemplated that other embodiments may be used with other training regimens, such as those used is sports such as swimming, football, baseball, softball, and the like.

The pacing device controller 108 may be housed in a box, or other suitable type of enclosure, having a sufficient size to house various components, such as a power source, control circuitry, and radio (e.g., transceiver) for communication with the wireless device 114. The exercise pacing device 100 may also include a signal conditioner 118 that is electrically coupled to the lights 106 at a specified distance from the pacing device controller 108 for amplifying control signals generated by the pacing device controller 108.

In one embodiment, the pacing device controller 108 may generate multiple moving indicators 110 that are spatially separated from one another and move along the elongated structure 102 at different speeds relative to one another. For example, the pacing device controller 108 may be configured to generate a first moving indicator 110 for a first athlete who starts at a first position along the elongated structure 102, and generate a second moving indicator 110 for a second athlete who starts at a second position along the elongated structure 102 so that they both can be trained using a single exercise pacing device 100. Additionally, the first moving indicator 110 may be configured to move along the elongated structure 102 at a different speed relative to the speed that the second moving indicator 110 moves.

In another embodiment, the pacing device controller 108 may generate other visual signals that convey visual information to the user. For example, the pacing device controller 108 may generate a blinking signal using certain lights 106 along the elongated structure 102 (e.g., the starting line of the elongated structure 102) that causes the lights to blink a specified quantity of times equal to a number of laps completed by the athlete. That is, the pacing device controller 108 may cause one or more lights 106 to blink once as the athlete completes one lap, blink twice when the athlete completes two laps, and so on as long as the athlete continues to complete additional laps along the elongated structure 102.

In one embodiment, the pacing device controller 108 may control the lights using an electrical cable extending along the elongated structure 102. For example, the electrical cable may include a ground wire, a power wire, and a clock wire that functions with a memory component, such as a SR flip-flop, a D flip-flop, or a JK flip-flop that is provided with each light 106 to sequentially energize the lights 106 along the elongated structure 102. Nevertheless, any suitable technique may be used to sequentially energize the lights 106 along the elongated structure 102 without departing from the spirit and scope of the present disclosure.

In some cases, one or more anchors 119 may be implemented to provide mechanical stability and keep the unit in place along the track. The anchors 119, which can be weights or other suitable mounting mechanisms, may be installed over the elongated structure 102. For example, weighted anchors may be used to hold the elongated structure 102 in place on a track that does not have preinstalled inner rails, while track rail anchors may be used to mount the elongated structure 102 to an existing track rail system.

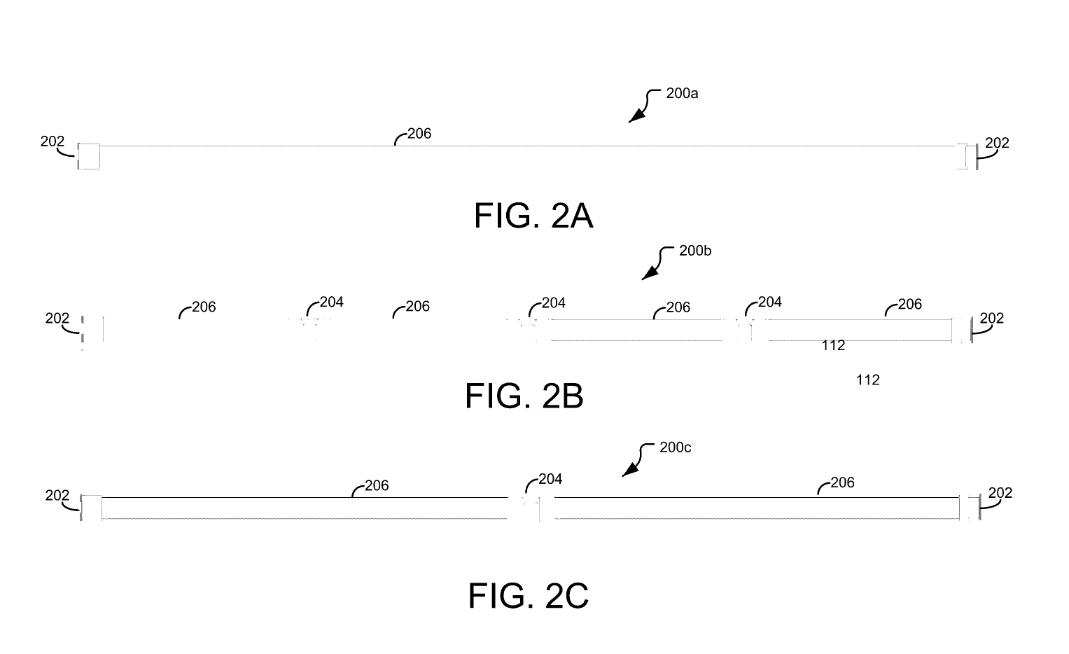

The elongated structure 102 may have any suitable size, shape, and/or structure that can be formed in a continuous loop, and can accommodate multiple lights 106 along its extent that can be controlled by a controller 108. In one embodiment, the elongated structure 102 comprises multiple modular elongated sections 200 (see FIGS. 2A, 2B, and 2C) that may be disposed adjacent one another to form a continuous loop. For example, the elongated sections 200 may be laid along the extent of an oval running track 116. Nevertheless, it is contemplated that the elongated structure 102 may be embodied in other forms. For example, the elongated structure 102 may be formed into a flexible cord, or other flexible structure, with lights (e.g., LEDs) configured on its outer shell in which the cord has a shape and flexibility sufficient to be wound on a reel when not in use and unreeled from the reel and formed into a continuous loop when being prepared for use.

FIGS. 2A, 2B, and 2C illustrate example modular elongated sections 200 that may be used to construct the elongated structure 102 according to one embodiment of the present disclosure. In general, elongated section 200a may be implemented along the curved section of a 400 meter oval running track, elongated section 200b may be implemented along the curved section of a 200 meter oval running track, while elongated section 200c may be implemented along the straight sections of both the 200 meter and the 400 meter oval running tracks. Although the elongated sections 200 are shown and described herein for use with 200 meter and 400 meter oval running tracks, it should be understood that other types and lengths of elongated sections may be implemented for use with other sizes and types of running tracks.

Each elongated section 200 includes extruded members 206 with electrical connectors 202 on each end for interconnection to adjacent elongated sections 200 configured at either end. Additionally, elongated sections 200a and 200b includes flex couplings 204 that join multiple extruded members 206 to allow its respective elongated section 200a and 200b to be bent to conform to the shape of the running track it is implemented on. To use, multiple elongated sections 200 are connected in series starting from the pacing device controller 108 and continuing around the track until it returns to form a continuous loop. Additionally, one or more signal conditioners 118 may be installed in line with the elongated sections 200 at specified intervals to amplify and condition timing signal transmitted from the pacing device controller 108.

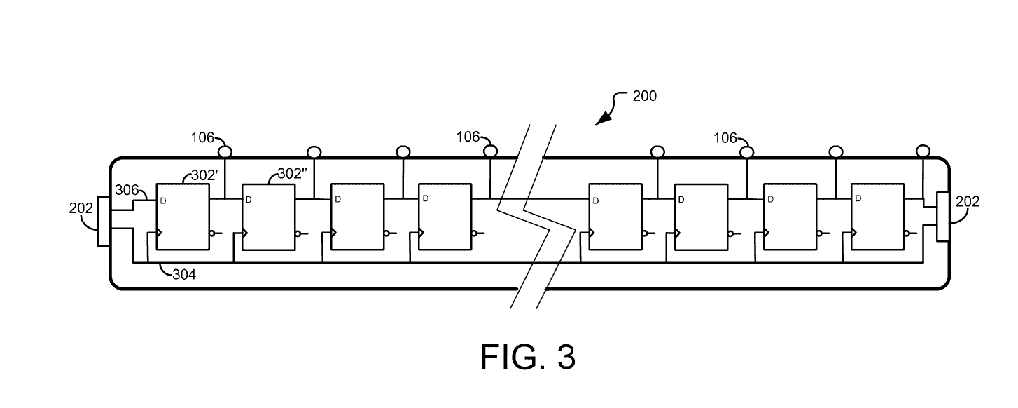

FIG. 3 illustrates an example elongated section 200 and associated circuitry that may be used to generate the moving indicator 110 through its respective section according to one embodiment of the present disclosure. The elongated section 200 may be the straight elongated section 200 as shown in FIG. 2A, the elongated section 200 having three flexing joints 204 as shown in FIG. 2B, the elongated section 200 having a single flexing joint 204 as shown in FIG. 2C, or another elongated section not shown herein.

In general, the circuitry includes multiple D flip-flops 302 arranged as a shift register in which the output of each flip-flop 302 is coupled to a light 106 arranged on the surface of the elongated section 200. Although only eight flip-flops 302 are shown, it should be appreciated that the elongated section 200 may include any quantity of flip-flops 302 and associated lights 106. For example, the elongated section 200 may have one hundred flip-flops 302 and associated lights 106 for generating the moving indicator 110.

In operation, the controller 108 generates a clock signal that is transmitted along a clock node 304 from a first connector 202, and a data signal that is sequentially transmitted along a data node 306 sequentially in daisy-chain fashion through the flip-flops 302. That is, when a logic high value is present at a first flip-flop 302′ during a first clock edge of the clock signal, the logic high value is propagated to the output of the first flip-flop 302′ for energizing the light 106 associated with the first flip-flop 302′. Since the output of the first flip-flop 302′ is also coupled to the second flip-flop 302″, a second clock edge of the clock signal will propagate the logic high value to the output of the second flip-flop 302″. The aforementioned process will continue throughout the extent of the elongated section such the logic high value is sequentially propagated through each flip-flop 302. Additionally, the clock signal and the data signal may be outputted through a second connector 202 for continuing the data signal and resulting moving indicator 110 to an adjacent elongated section 200.

In one embodiment, the controller 108 may generate a moving indicator 110 in which multiple adjacent lights are simultaneously lit. For example, the controller 108 may generate a data signal with a pulse with for an elapsed period of time that is approximately 10 times that of the clock signal such that the moving indicator 110 comprises 10 adjacent lights that are simultaneously lit as it moves along the elongated section 200. Nevertheless, it should be appreciated that the controller 108 may generate the moving indicator 110 to have any desired quantity of simultaneously lit lights 106, such as only 1 simultaneously lit light, less than 10 simultaneously lit lights, or greater than 10 simultaneously lit lights without departing from the spirit and scope of the present disclosure.

Although the elongated section 200 shows on example of a circuit that may be implemented to generate the moving indicator 110, it should be appreciated that other types of circuitry may be implemented for generating the moving indicator 110 without departing from the spirit and scope of the present disclosure. For example, it is contemplated that the elongated section 200 may include an embedded processing device including a processor that executes instructions stored in a memory, or other suitable logic state machine, for receiving signals from the first connector 202 to generate individual signals to each light 106 for generating the moving indicator 110.

The elongated section 200 may also include additional, different, or fewer components that what is shown herein. For example, the elongated section 200 may include one or more buffer components between the output of each flip-flop 302 and the lights 106 to provide additional drive current to each light 106. As another example, the elongated section 200 may also include one or more noise cancelling devices, such as capacitors, inductors, and the like, for mitigating any noise (e.g., electro-magnetic interference (EMI)) that may be superimposed on either the clock signal or the data signal.

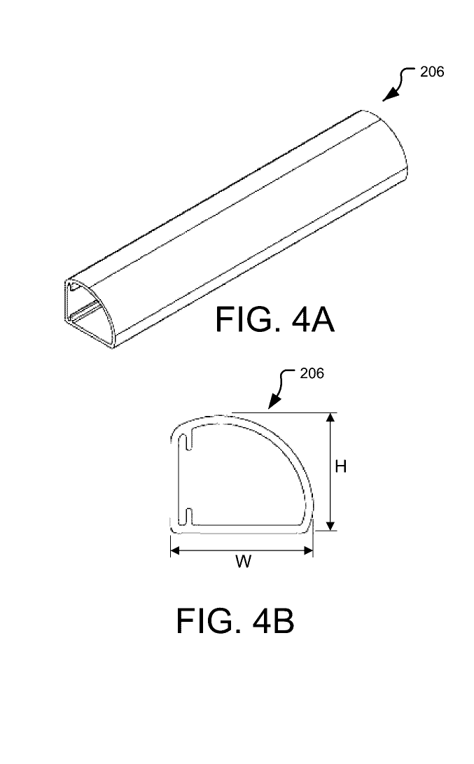

FIGS. 4A and 4B illustrate a perspective view and end view, respectively, of an extruded member 206 according to one embodiment of the present disclosure. As shown, the extruded member 206 has a height H of approximately 1.116 inches and a width W of approximately 1.32 inches. Although example dimensions are provided for the length and cross-sectional size of the elongated sections 200, it should be understood that elongated sections of other lengths and cross-sectional shapes may be implemented without departing from the spirit and scope of the present disclosure.

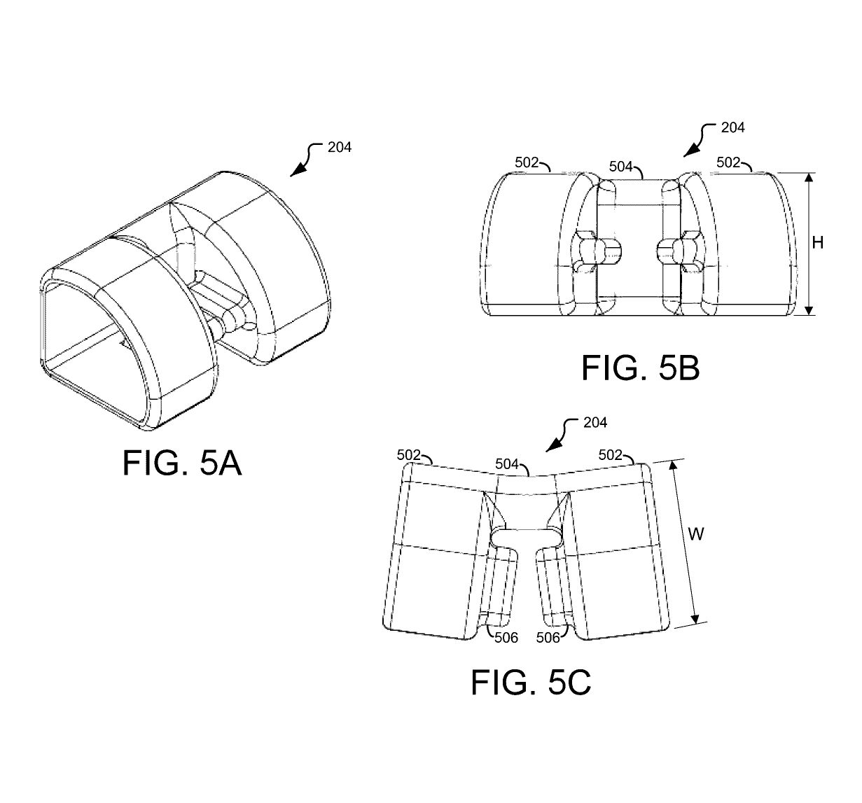

FIGS. 5A, 5B, and 5C illustrate a perspective view, a side view, and a top view, respectively, of a flex coupling 204 according to one embodiment of the present disclosure. As shown, the extruded member 206 has a height H of approximately 1.354 inches and a width W of approximately 1.58 inches. Although example dimensions are provided for the length and cross-sectional size of the elongated sections 200, it should be understood that elongated sections of other lengths and cross-sectional shapes may be implemented without departing from the spirit and scope of the present disclosure.

The flex coupling 204 includes two receptacle portions 502 for receiving ends of an extruded member 206 and a living hinge portion 504 integrally formed with both receptacle portions 502. Projections 506 are formed on each receptacle portions 502 and function to inhibit flexure of the living hinge 504 to only one direction. Thus, the projections may serve to ensure that the elongated structure 200 can be efficiently laid out in a straight orientation if so desired.

The extruded members 206, connectors 202, flex couplings 204, and extruded members 206 may be formed of any suitable material, such as plastic, sheet metal, or other suitable substance. In one example the extruded members 206 may comprise extruded sections of plastic that are cut to the desired length with flex couplings 204 and connectors 202 attached at specified positions along the extruded section 206.

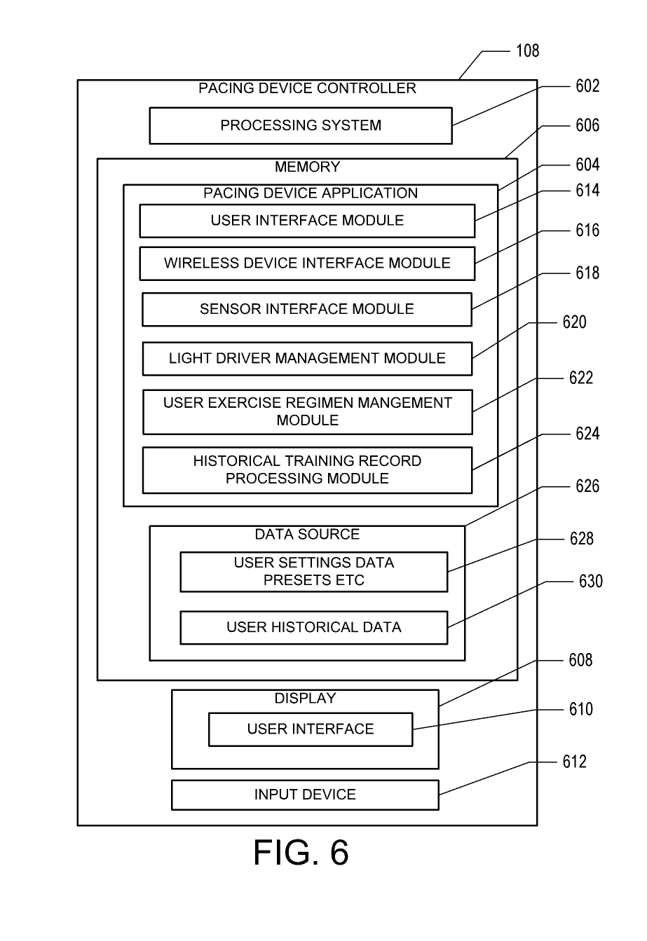

FIG. 6 illustrates one example of the pacing device controller 108 that may be used to control the operation of the exercise pacing device 100 according to one embodiment of the present disclosure. The pacing device controller 108 includes a processing system 602 that executes a pacing device application 604 stored in a memory 606 (e.g., computer readable media). Although the pacing device controller 108 is shown and described as a computer-based design incorporating instructions stored in a memory 606 and executed by a processor 602, it should be understood that the pacing device controller 108 may be embodied in other specific forms, such as using discrete and/or integrated analog circuitry, field programmable gate arrays (FPGAs), application specific integrated circuitry (ASICs), or any combination thereof. Additionally, the pacing device controller 108 may be omitted if manual operation of the exercise pacing device 100 is desired.

The processing system 602 includes one or more processors or other processing devices and memory. The one or more processors may process machine/computer-readable executable instructions and data, and the memory may store machine/computer-readable executable instructions and data including one or more applications, including the application 604. A processor is hardware and memory is hardware. The memory 606 includes random access memory (RAM) and/or other non-transitory memory, e.g., a non-transitory computer-readable medium such as one or more flash disks or hard drives. The non-transitory memory may include any tangible computer-readable medium including, for example, magnetic and/or optical disks, flash drives, and the like.

The pacing device controller 108 may also include a display 608, such as a liquid crystal display (LCD), one or more light emitting diodes (LEDs), an LED display, a touch screen, a capacitive display, or another display for displaying configuration settings associated with the application 604. The pacing device controller 108 may include a user interface 610 that may be displayed on the display 608 for providing information to the user as well as receiving user input for manipulating the operation of the application 604. The pacing device controller 108 may also include an input device 612, such as one or more buttons, switches, or other electro-mechanical device for providing user input to the application 604. In one example, the display 608 and input device 612 may include a touch screen display for receiving user input and displaying one or more characteristics associated with operation of the application 604. In one embodiment, the display 608 may include a user interface 610 for displaying information to the user, and receiving user input from the user. In another example, the display 608 may have a size sufficient for displaying information, such as user performance data (e.g., lap time, lap quantity, pacing accuracy, etc.) that is readable by the user, while the user is being paced around the elongated structure 102 by the moving indicator 110.

In general, the processing system 602 executes an exercise pacing device application 604 with one or more modules to control the operation of the exercise pacing device 100. In certain embodiments, the application 604 may control all operating aspects of the exercise pacing device 100 described herein. In other embodiments, the application 604 may control only one or a subset of the operating aspects of the exercise pacing device 100 described herein. For example, in certain embodiments, the application 604 may control certain aspects of the exercise training device 100, while the wireless device 114 controls other aspects of the exercise training device 100.

A user interface module 614 facilitates the receipt of user data and/or other communications from the input device 610 of the pacing device controller 108. In one embodiment, the pacing device controller 108 generates and executes the user interface 612 that displays an interactive display such as the display 608, or other suitable user interface mechanism including one or more selectable fields, editing screens, and the like for displaying status information associated with one or more aspects of the application 604, such as operational status information, power source (e.g., battery) condition, and the like.

A wireless device interface module 616 communicates with the wireless device 114 to facilitate the receipt of user data from the wireless device 114 for manipulating the operation of the pacing device controller 108 and/or transmitting information (e.g., user performance data, operational status of the pacing device controller 108, etc.) to the wireless device 114 for display by the wireless device 114. Either of the user interface module 614 and the wireless device interface module 616 may receive and process none, some, or all information for manipulating the operation of the pacing device controller 108 and/or display information for view by the user. For example, the pacing device controller 108 may be void of any display 608 and/or input device 612 such that most or all user interaction and display of information is provided through the wireless device 114.

A sensor interface module 618 monitors the various sensors 112 configured on the exercise pacing device 100 and generates signals to be used by the one or more other modules of the application 604 for administering the operation of the exercise pacing device 100. For example, the sensor interface module 616 may continually monitor one or more user proximity sensors 112 (e.g., radio frequency identification (RFID), passive infrared sensor (PIR) sensors, etc.) to detect the location of a user and transmits signals to be used by the user exercise regimen management module 622 for correlating the location of the user relative to the moving indicator 110. As another example, the sensor interface module 618 may communicate with one or more telemetry sensors (e.g., Bluetooth sensors) to receive telemetry information from a device worn by the user, such as a heart rate monitor that can be used to correlate strenuousness of a training regimen to the heart rate of the user.

A light driver management module 620 controls the operation of the lights 106 along the elongated structure 102 to generate the moving indicator 110. Any suitable technique may be used for controlling the operation of the lights 106. For example, the light driver management module 620 may generate a clocking signal and a data signal that may be used by one or more shift registers included in the elongated structure 102 to mimic the moving indicator 110. (See FIG. 3). For another example, the light driver management module 620 may generate signals to be independently transmitted to each of multiple wirelessly controlled lights configured around the continuous loop. For such a structure, a suitable wireless control medium (e.g., Bluetooth signaling, etc.) may be used for conveying the signals from the light driver management module 620 to each wirelessly controlled lights.

A user exercise regimen management module 622 receives control signals from the user interface module 614 and/or the wireless device interface module 616, and processes the received control signals to manage the operation of the light driver management module 620. For example, the user exercise regimen management module 622 may receive signal from the user interface module 614 for establishing a lap pacing regimen in which the moving indicator 110 is to complete a set of 10 laps in which the moving indicator 110 completes each lap at approximately 1 minute and 20 seconds. Thus, the user exercise regimen management module 622 may calculate a clocking frequency such that the moving indicator completes each lap via the circumference of the elongated structure at 1 minute and 20 seconds and stops once 10 laps have been completed.

In one embodiment, the user exercise regimen management module 622 may be configured to generate a pacing regimen that changes over a particular training session. For example, the user exercise regimen management module 622 may be programmed to generate a training session having 20 laps in which the moving indicator 110 is to complete the first 5 laps at 1 minute and 40 seconds, the second 5 laps at 1 minute and 20 seconds, the third 5 laps at 1 minute and 5 seconds, and the fourth 5 laps at 1 minute and 40 seconds. Thus, the user exercise regimen management module 622 may adjust the speed of the moving indicator 110 to initially pace the user slowly, gradually increase speed over the course of the training regimen, and slow the pace of the moving indicator 110 at the end of the training regimen.

As another example, the user exercise regimen management module 622 may generate another training regimen commonly referred to as interval training in which the pace of the moving indicator 110 may be changed over differing portions of the elongated structure 102. In such a case, the user exercise regimen management module 622 may pace the moving indicator 110 along the elongated structure 102 at a sprint speed over a certain portion of the track (e.g., the straight portions of the track), and pace the moving indicator 110 along the elongated structure 102 at a walking speed over other portions of the track (e.g., the curved portions of the track).

Once the training regimens are received and processed by the user exercise regimen management module 622, they may be stored as user setting data 628 in the data source 626 for use at a later time. For example, the user exercise regimen management module 622 may receive and process information from the user for generating an interval training regimen, and store the received and processed information in the user setting data 628. At a later point in time, the user exercise regimen management module 622 may receive instructions for loading that interval training regimen from the user settings data 628 so that the interval training regimen may be used by the user. Additionally, certain settings associated with the interval training regimen may be changed by the user, such as the relative spring speeds and/or walking speeds to be implemented with the interval training regimen.

A historical training record processing module 624 receives information associated with completed pacing regimens, performs one or more processes on the received information, and stores the processes information in the data source 626 as user historical data 630. For example, the historical training record processing module 624 may receive pacing regimen information from the user exercise regimen management module 622 once a pacing regimen has been completed by the user, process this information, such as organize the information into easily storable form, extract useful characteristics (e.g., lap speed, lap completion efficiency, overall pacing regimen completion times, etc.) from the information, and/or infer additional information using certain sensors (e.g., obtaining location data obtained from proximity sensors to correlate pacing efficiency relative to the moving position of the moving indicator 110, etc.).

In one embodiment, the historical training record processing module 624 may correlate multiple pacing regimens that may have been performed by a user over an extended period of time to derive additional information about the pacing regimens performed by the user. For example, once the historical training record processing module 624 obtains and stores information associated with multiple training sessions that have been performed by the user over an extended period of time (e.g., one training session each day for a week), one or more processing techniques may be performed on the information to derive additional information that may be useful to the user, such as improvements in performance, changes in performance due to one or more external factors (e.g., changes in diet of the user, lifestyle habits of the user, etc.). Thus, the historical training record processing module 624 may be used to derive additional information that would otherwise not be available to the user for assessing a current pacing performance of the user, and any changes that can be made to enhance the pacing performance of the user over time.

It should be appreciated that the modules described herein are provided only as an example of a computing device that may execute the application 604 according to the teachings of the present invention, and that other computing devices may have the same modules, different modules, additional modules, or fewer modules than those described herein. For example, one or more modules as described in FIG. 6 may be combined into a single module. As another example, certain modules described herein may be encoded and executed on other circuits, such as another circuit that is separate from the pacing device controller 108.

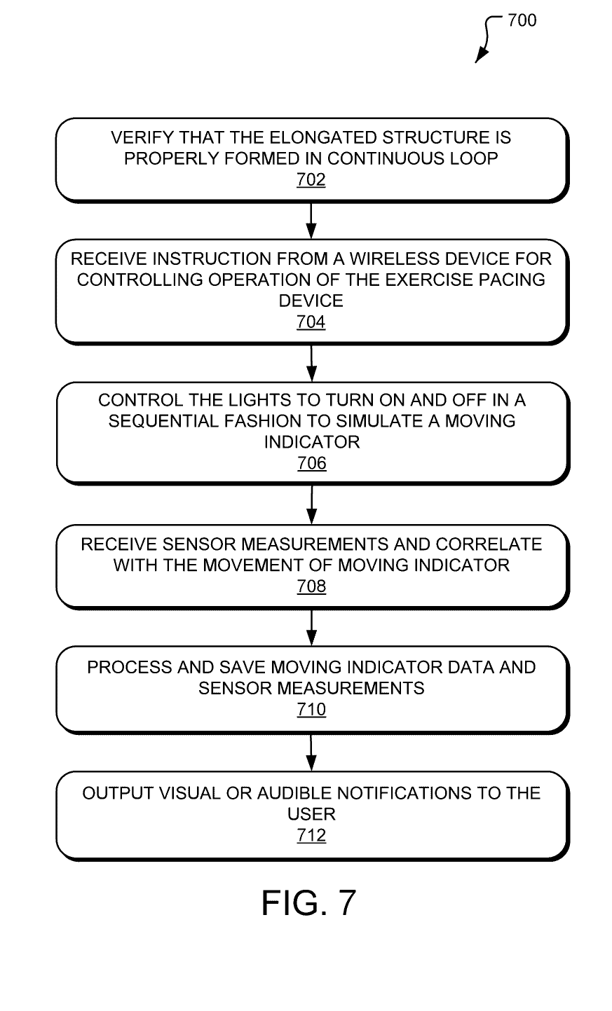

FIG. 7 illustrates an example process that may be performed by the exercise pacing device 100 according to one embodiment of the present disclosure. Initially, the elongated structure 102 is disposed in a continuous loop at a desired location, such as along a perimeter of a running track, such as a 200 meter or a 400 meter running track. In one embodiment, the elongated structure 102 may be formed from multiple modular elongated sections 200 that are disposed adjacent one another to form the continuous loop in which the modular elongated sections form the elongated structure. In another embodiment, the elongated structure 102 may include a flexible elongated section of cord material that can be rolled onto a reel for storage, and unrolled to form a continuous loop. In yet another embodiment, the exercise pacing device 100 may include multiple wireless lighting structures that can be placed around a continuous loop, such as a running track, in which each wireless lighting structure wirelessly receives instructions from the controller 108 for alternatively turning a light on and off to simulate a moving indicator around the continuous loop.

At step 702, the pacing device controller 108 optionally verifies that the elongated structure has been properly formed into a continuous loop. To this end, the pacing device controller 108 may include a first connector to be mated to a complementary first connector configured at a first end of the elongated structure 102, and a second connector to be mated to a second complementary connector at a second end of the elongated structure 102. When the pacing device controller 108 is initially powered on, it may transmit a test signal into the elongated structure 102 at the first connector and verify that the test signal is received at the other end at its second connector. If the test signal is not properly received, the pacing device controller 108 may generate an alarm to alert the user that the elongated structure 102 is not properly configured.

At step 704, the pacing device controller 108 receives instructions for operation of the exercise pacing device 100. The instructions may be received from a remotely configured wireless device 114, a user interface configured on the controller 108 or a combination of the two. The instructions may include, for example, setting a starting location along the continuous loop, a speed at which the moving indicator (e.g., a rabbit) moves around the continuous loop, a quantity of revolutions (e.g., laps) that the moving indicator 110 moves around the elongated structure 102, and/or any changes to the speed of the moving indicator (e.g., interval training loops). In one embodiment, the pacing device controller 108 may receive instructions for generating two or more independent moving indicators for a corresponding two or more users (e.g., athletes).

In one embodiment, the pacing device controller 108 may store the received instructions such that they may be retrieved from memory for use over future training sessions. For example, a first training regimen for a first user may include instructions for establishing a speed of 1 minute, 30 seconds of the moving indicator 110 for 20 laps, and a second training session for a second user to include instructions for establishing a speed of 1 minute, 10 seconds of the moving indicator 110 for 10 laps. Each of these training sessions may be independently stored in the pacing device controller 108 so that on ensuing training sessions, the first and/or the second training session may be independently loaded from memory without the need for re-entry of the instructions each time the system 100 is used.

At step 706, the pacing device controller 108 controls the lights to turn on and off in a sequential fashion along the continuous loop of the elongated structure. The lights may be turned on and off at a speed sufficient to simulate a moving indicator for pacing a user around the continuous loop. While the moving indicator 110 is generated along the elongated structure 102, the pacing device controller 108 may also receive sensor measurements from one or more sensors 112 configured along the elongated structure 102 at step 708. For example, the pacing device controller 108 may receive proximity information from each of multiple proximity sensors (e.g., RFID sensors, PIR sensors, etc.) that indicates a location of the user as the user walks, runs or otherwise ambulates around the elongated structure 102. As another example, the pacing device controller 108 may receive telemetry information from a device worn by the user, such as a heart rate monitor that conveys heart rate information to the pacing device controller 108.

In one embodiment, one or more signal conditioners 118 may be employed at various intervals along the continuous loop for conditioning the signals along the continuous loop. For example, the signal conditioners 118 may be used to amplify signals generated by the pacing device controller 108, and/or clean up various timing characteristics of the signals, such as filtering of flutter, re-synchronizing multiple signals, or removing parasitic impulse noises from the signals.

The steps described above may be repeated throughout the use of the exercise pacing device 100. For example, after a total of 4 laps have been completed using a first set of instructions received from a remote wireless device 114, the pacing device controller 108 may receive one or more additional instructions, such as one in which the user wishes to increase the speed of the moving indicator 110. Thus in this case, the pacing device controller 108 may continue its operation, with the moving indicator 110 moving at the new speed setting received from the wireless device 114.

At step 710, the pacing device controller 108 processes and saves training session information along with any received sensor information in the memory 606 for review at a later time. For example, the pacing device controller 108 may process known information about the location and movement of the moving indicator 110 with proximity information associated with a determined location of the user to derive certain characteristics about how well the user performed the training session. As another example, the pacing device controller 108 may process known information about the location and movement of the moving indicator 110 with heart rate information obtained from one or more telemetry sensors to correlate a heart rate of the user with their movement about the continuous loop. Additionally, the pacing device controller 108 may process the information from multiple training sessions (e.g., a month long training regimen) to provide a report that may indicate, among other things, increases in performance that have been gained over the multiple training sessions, and/or lapses in performance that may have been incurred over the multiple training sessions due to various factors, such as dietary habits, bodily injury, emotional distress, and the like.

In one embodiment, the pacing device controller 108 may automatically adjust a speed of the moving indicator 110 according to an obtained heart rate of the user. For example, the pacing device controller 108 may receive instructions for maintaining a specified heart rate such that, when the training session is conducted, the pacing device controller 108 may obtain ongoing (e.g., periodic) measurements of the user's heart rate, and decrease the speed of the moving indicator 110 when the user's heart rate goes above an upper threshold value and increase the speed of the moving indicator 110 when the user's heart rate goes below a lower threshold value.

At step 712, the pacing device controller 108 outputs notification information to the user. For example, the display 608 of the pacing device controller 108 may have a size sufficiently large to enable view by the user while the user is using the device (e.g., running along with the moving indicator 110) such that the display 608 may include information useful to the user, such as a quantity of completed laps, how well the movement of the user coincides with the movement of the moving indicator 110, a measured heart rate of the user, and the like. For another example, the pacing device controller 108 may provide other forms of information for the user, such as alternatively flashing the moving indicator 110 a number of times corresponding to how many laps around the elongated structure 102 have been completed.

The above process is repeatedly performed during use of the pacing device controller 108. Nevertheless, when use of the pacing device controller 108 is no longer needed or desired, the process ends.

Although FIG. 7 describes one example of a process that may be performed by the pacing device controller 108 to generate a moving indicator 110 according to instructions from a user, the features of the disclosed process may be embodied in other specific forms without deviating from the spirit and scope of the present disclosure. For example, the pacing device controller 108 may perform additional, fewer, or different operations than those operations as described in the present example. As another example, the steps of the process described herein may be performed by a computing system other than the computing system 102, which may be, for example, a mobile application (e.g., an app) executed on the wireless device 114. For yet another example, the identified steps of the process may be performed in any suitable order; that is, the process as described above is not restricted to the sequence in which those have been described.

The description above includes example systems, methods, techniques, instruction sequences, and/or computer program products that embody techniques of the present disclosure. However, it is understood that the described disclosure may be practiced without these specific details.

In the present disclosure, the methods disclosed may be implemented as sets of instructions or software readable by a device. Further, it is understood that the specific order or hierarchy of steps in the methods disclosed are instances of example approaches. Based upon design preferences, it is understood that the specific order or hierarchy of steps in the method can be rearranged while remaining within the disclosed subject matter. The accompanying method claims present elements of the various steps in a sample order, and are not necessarily meant to be limited to the specific order or hierarchy presented.

The described disclosure may be provided as a computer program product, or software, that may include a machine-readable medium having stored thereon instructions, which may be used to program a computer system (or other electronic devices) to perform a process according to the present disclosure. A machine-readable medium includes any mechanism for storing information in a form (e.g., software, processing application) readable by a machine (e.g., a computer). The machine-readable medium may include, but is not limited to, magnetic storage medium (e.g., floppy diskette), optical storage medium (e.g., CD-ROM); magneto-optical storage medium, read only memory (ROM); random access memory (RAM); erasable programmable memory (e.g., EPROM and EEPROM); flash memory; or other types of medium suitable for storing electronic instructions.

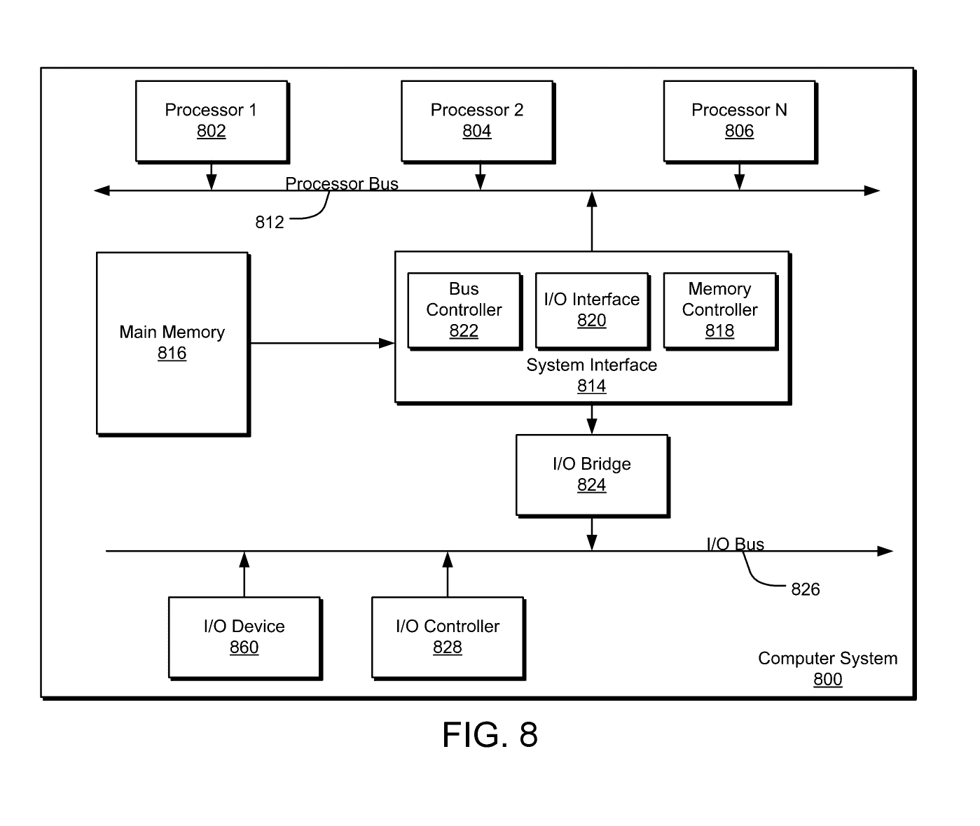

For example, FIG. 8 is a block diagram illustrating an example of a host or computer system 800 which may be used in implementing the embodiments of the present disclosure. The computer system (system) includes one or more processors 802-806. Processors 802-806 may include one or more internal levels of cache (not shown) and a bus controller or bus interface unit to direct interaction with the processor bus 812. Processor bus 812, also known as the host bus or the front side bus, may be used to couple the processors 802-806 with the system interface 814. System interface 814 may be connected to the processor bus 812 to interface other components of the system 800 with the processor bus 812. For example, system interface 814 may include a memory controller 818 for interfacing a main memory 816 with the processor bus 812. The main memory 816 typically includes one or more memory cards and a control circuit (not shown). System interface 814 may also include an input/output (I/O) interface 820 to interface one or more I/O bridges or I/O devices with the processor bus 812. One or more I/O controllers and/or I/O devices may be connected with the I/O bus 826, such as I/O controller 828 and I/O device 860, as illustrated.

I/O device 860 may also include an input device (not shown), such as an alphanumeric input device, including alphanumeric and other keys for communicating information and/or command selections to the processors 802-806. Another type of user input device includes cursor control, such as a mouse, a trackball, or cursor direction keys for communicating direction information and command selections to the processors 802-806 and for controlling cursor movement on the display device.

System 800 may include a dynamic storage device, referred to as main memory 816, or a random access memory (RAM) or other computer-readable devices coupled to the processor bus 812 for storing information and instructions to be executed by the processors 802-806. Main memory 816 also may be used for storing temporary variables or other intermediate information during execution of instructions by the processors 802-806. System 800 may include a read only memory (ROM) and/or other static storage device coupled to the processor bus 812 for storing static information and instructions for the processors 802-806. The system set forth in FIG. 8 is but one possible example of a computer system that may employ or be configured in accordance with aspects of the present disclosure.

According to one embodiment, the above techniques may be performed by computer system 800 in response to processor 802-806 executing one or more sequences of one or more instructions contained in main memory 816. These instructions may be read into main memory 816 from another machine-readable medium, such as a storage device. Execution of the sequences of instructions contained in main memory 816 may cause processors 802-806 to perform the process steps described herein. In alternative embodiments, circuitry may be used in place of or in combination with the software instructions. Thus, embodiments of the present disclosure may include both hardware and software components.

A computer readable medium includes any mechanism for storing or transmitting information in a form (e.g., software, processing application) readable by a machine (e.g., a computer). Such media may take the form of, but is not limited to, non-volatile media and volatile media. Non-volatile media includes optical or magnetic disks. Volatile media includes dynamic memory, such as main memory 816. Common forms of machine-readable medium may include, but is not limited to, magnetic storage medium (e.g., hard disk drive); optical storage medium (e.g., CD-ROM); magneto-optical storage medium; read only memory (ROM); random access memory (RAM); erasable programmable memory (e.g., EPROM and EEPROM); flash memory; or other types of medium suitable for storing electronic instructions.

Embodiments of the present disclosure include various operations or steps, which are described in this specification. The steps may be performed by hardware components or may be embodied in machine-executable instructions, which may be used to cause a general-purpose or special-purpose processor programmed with the instructions to perform the steps. Alternatively, the steps may be performed by a combination of hardware, software and/or firmware.

It is believed that the present disclosure and many of its attendant advantages will be understood by the foregoing description, and it will be apparent that various changes may be made in the form, construction, and arrangement of the components without departing from the disclosed subject matter or without sacrificing all of its material advantages. The form described is merely explanatory, and it is the intention of the following claims to encompass and include such changes.

While the present disclosure has been described with reference to various embodiments, it will be understood that these embodiments are illustrative and that the scope of the disclosure is not limited to them. Many variations, modifications, additions, and improvements are possible. More generally, embodiments in accordance with the present disclosure have been described in the context of particular implementations. Functionality may be separated or combined in blocks differently in various embodiments of the disclosure or described with different terminology. These and other variations, modifications, additions, and improvements may fall within the scope of the disclosure as defined in the claims that follow.

What is claimed is:

1. An exercise pacing system comprising:
an elongated structure having two ends that are configured to be disposed adjacent one another to form a continuous loop;
a plurality of lights configured at regular intervals along the elongated structure; and
a controller comprising at least one memory for storing an application that when executed by at least one processor, is executed to control the lights to turn on and off along the continuous loop of the elongated structure; wherein the lights are turned on and off at a speed sufficient to simulate a moving indicator.

2. The exercise pacing system of claim 1, wherein the elongated structure comprises a plurality of modular elongated sections that may be disposed adjacent one another to form the continuous loop, wherein the controller is executed to control the lights using an electrical cable configured in each of the elongated sections, the electrical cable of adjacent sections coupled together using a connector at both ends of the modular elongated sections.

3. The exercise pacing system of claim 1, further comprising a signal conditioner coupled to the lights at a specified distance from the controller, the signal conditioner configured to amplify signals generated by the controller for controlling the lights.

4. The exercise pacing system of claim 1, wherein the controller is further executed to receive instructions from a user interface for controlling the operation of the moving indicator, wherein the instructions comprise at least one of a speed of the moving indicator, a quantity of revolutions to be completed by the moving indicator, and a quantity of moving indicators to be simultaneously generated by the controller.

5. The exercise pacing system of claim 4, wherein the controller is further executed to receive the instructions via wireless signals from a wireless device for controlling the operation of the controller, the wireless device comprising the user interface.

6. The exercise pacing system of claim 4, wherein the user interface is configured on the controller.

7. The exercise pacing system of claim 4, wherein the controller is further executed to modify a speed of the moving indicator when the moving indicator arrives at one or more specified positions along the elongated structure, the modification of the speed and the positions where the speed is changed are set according to the received instructions.

8. The exercise pacing system of claim 4, wherein the controller is further configured to receive proximity information associated with a location of the user and generate a report indicating how well the a position of the user coincided with the position of the moving indicator.

9. The exercise pacing system of claim 4, wherein the controller is further configured to receive telemetry information associated with one or more characteristics of the user, and generate a report correlating the telemetry information with the movement of the moving indicator.

10. The exercise pacing system of claim 9, wherein the telemetry information comprises a measured heart rate of the user, the controller further executed to adjust a speed of the moving indicator according to the measured heart rate of the user.

11. An exercise pacing method comprising:
disposing two ends of an elongated structure adjacent to one another to form a continuous loop, the elongated structure comprising a plurality of lights configured at regular intervals along the elongated structure; and
controlling, using a set instructions stored in at least one memory and executed by at least one processor, the lights to turn on and off along the continuous loop of the elongated structure; wherein the lights are turned on and off at a speed sufficient to simulate a moving indicator.

12. The exercise pacing method of claim 11, further comprising receiving instructions from a user interface for controlling the operation of the moving indicator, wherein the instructions comprise at least one of a speed of the moving indicator, a quantity of revolutions to be completed by the moving indicator, and a quantity of moving indicators to be simultaneously generated by the controller.

13. The exercise pacing method of claim 12, further comprising receiving the instructions via wireless signals from a wireless device for controlling the operation of the controller, the wireless device comprising the user interface.

14. The exercise pacing method of claim 12, further comprising modifying a speed of the moving indicator when the moving indicator arrives at one or more specified positions along the elongated structure, the modification of the speed and the positions where the speed is changed are set according to the received instructions.

15. The exercise pacing method of claim 12, further comprising receiving proximity information associated with a location of the user and generate a report indicating how well the a position of the user coincided with the position of the moving indicator.

16. The exercise pacing method of claim 12, further comprising receiving telemetry information associated with one or more characteristics of the user, and generating a report correlating the telemetry information with the movement of the moving indicator.

17. The exercise pacing method of claim 16, further comprising adjusting a speed of the moving indicator according to the measured heart rate of the user, the measured heart rate comprising one of the one or more characteristics.

18. An exercise pacing device comprising:
a plurality of lights configured to be placed at regular intervals along a running track; and
a controller comprising at least one memory for storing an application that when executed by at least one processor, is executed to control the lights to turn on and off along the continuous loop of the elongated structure; wherein the lights are turned on and off at a speed sufficient to simulate a moving indicator for pacing a user around the running track.

19. The exercise pacing device of claim 18, further comprising an elongated structure having two ends that are configured to be disposed adjacent one another to form a continuous loop around the running track, wherein the elongated structure comprises a plurality of modular elongated sections that may be disposed adjacent one another to form the continuous loop, wherein the controller is executed to control the lights using an electrical cable configured in each of the elongated sections, the electrical cable of adjacent sections coupled together using a connector at both ends of the modular elongated sections.

20. The exercise pacing device of claim 19, wherein the controller is further executed to receive instructions from a user interface configured on a wireless device for controlling the operation of the moving indicator, wherein the instructions comprise at least one of a speed of the moving indicator, a quantity of revolutions to be completed by the moving indicator, and a quantity of moving indicators to be simultaneously generated by the controller.
